SEQUENCE LISTING (1 sequence)

SEQ ID NO: 1 - protein, 13 aa, artificial sequence
  primer_bind: TSP-517 peptide from thrombospondin
KRAKQAGWSHWAA

(19) United States
(12) Patent Application Publication
(10) Pub. No.: US 2005/0008572 A1
(43) Pub. Date: Jan. 13, 2005

(54) Nanoparticular tumor targeting and therapy

(76) Inventors: Ales Prokop, Nashville, TN (US); Jeffrey M. Davidson, Nashville, TN (US); Gianluca Carlesso, Nashville, TN (US); David Roberts, Bethesda, MD (US)

Correspondence Address: Dr. Benjamin Adler;ADLER & ASSOCIATES, 8011 Candle Lane, Houston, TX, 77071, US

(21) Appl. No.: 10/833,370

(22) Filed: Apr. 28, 2004

Related U.S. Application Data
(60) Provisional application No. 60/466,375, filed on Apr. 29, 2003.

(51) Int. Cl. A61K 9/14; A61K 49/00

(52) U.S. Cl. 424/9.6; 424/489

(57) ABSTRACT

The present invention provides a series of biocompatible, nanoparticulate formulations that are designed to retain and deliver peptides such as anti-angiogenic factors over an extended time course. The nanoparticles can be targeted to a cell or tissue by targeting ligands crosslinked or conjugated to the corona of the nanoparticles. In addition to selective targeting, the nanoparticles also may perform noninvasive imaging using bioluminescence and/or magnetic resonance imaging via a contrast agent in the core of the nanoparticle. Also provided are methods of delivering to and, optionally, imaging of a cell or tissue. Furthermore, methods of producing the nanoparticles in batch or continous mode via simple mixing or micromixing.

### CROSS-REFERENCE TO RELATED APPLICATIONS

This non-provisional application claims benefit of provisional U.S. Ser. No. 60/466,375, filed Apr. 29, 2003, now abandoned.

### BACKGROUND OF THE INVENTION

### FEDERAL FUNDING LEGEND

This invention was produced in part using funds obtained through grant 5R21HL065982 from the National Institutes of Health. Consequently, the federal government has certain rights in this invention.

### FIELD OF THE INVENTION

The present invention relates generally to the field of cancer therapy.

More specifically, the present invention provides a nanoparticle delivery system capable of targeting tumor vasculature and delivering anti-angiogenic compounds.

### DESCRIPTION OF THE RELATED ART

Development of therapies aimed at inhibiting the growth of new blood vessels is among the most intensively studied approaches in the treatment of cancer (1-2).

Since the first mention of tumor vasculature as a potential therapeutic target 30 years ago, understanding of the intricate mechanisms leading to the formation of new blood vessels associated with tumor growth and the spread of metastases has greatly improved (3).

This research has led to the discovery of numerous regulatory molecules that influence endothelial cell physiology in vitro and angiogenesis in vivo. They can be divided into two groups: angiogenic factors consisting of growth factors, extracellular matrix molecules and their membrane-bound proteins, e.g. integrins, growth factor receptors, and anti-angiogenic substances.

The anti-angiogenic molecules are believed to have promising applications in the therapy of cancer, arthritis and ocular neovascularization. There are currently more than 30 angiogenesis inhibitors in clinical trials (2), and a multitude of promising new candidates are under investigation in vitro and in animal models. An important therapeutic strategy is the exploitation of endogenous anti-angiogenic molecules to inhibit further tumor growth, to avoid tumor spread and establishment of new distant metastases, or even to shrink the tumor, together with low side effects. Current data demonstrate that tumors and, by inference, capillaries regress when exposed to fragments of plasminogen, i.e., angiostatin, type XVIII collagen, i.e., endostatin and peptides derived from thromobospondin-1 (TSP-1) (4-7).

Thrombospondin-1 is a large trimeric glycoprotein composed of three identical 180 kd subunits linked by disulfide bonds. The majority of anti-angiogenic activity is found in the central stalk region of this protein. There are at least two different structural domains within this central stalk region that inhibit neovascularization. Besides TSP-1, there are six other proteins, i.e., fibronectin, laminin, platelet factor-4, angiostatin, endostatin and prolactin fragment, in which peptides have been isolated that inhibit angiogenesis. In addition, the dominant negative fragment of Flk1 and analogues of the peptide somatostatin are known to inhibit angiogenesis.

Endostatin is a 20 kDa protein fragment of collagen XVIII. It is a potent inhibitor of tumor angiogenesis and tumor growth (6). Angiostatin is a 38 kDa polypeptide fragment of plasminogen. Whereas plasminogen has no fibrinogenic activity, angiostatin has marked angiogenic activity (4). Angiostatin was isolated when it was observed that the primary tumor suppressed metastases. That is, when the primary tumor was removed, the metastases grew. Administration of angiostatin blocks neo-vascularization and growth of metastases.

The Flk1 receptor is a receptor for vascular endothelial growth factor (VEGF). Flk-1 is expressed exclusively on the surface of the endothelial cells. Once VEGF binds to the receptor, the Flk-1 receptor then homodimerizes to stimulate the endothelial cell to divide. If a mutant receptor of Flk-1 is transfected into the endothelial cells, the mutant receptor dimerizes with the wild-type Flk-1 receptor. In endothelial cells transfected with the mutant Flk-1 receptor, VEGF is unable to stimulate the endothelial cells to divide. Co-administration of a retrovirus carrying the Flk-1 cDNA inhibits tumor growth. This emphasizes that the receptor plays a critical role in the angiogenesis of solid tumors.

Chemotherapeutic drugs are often highly toxic and this places a limit on the dose that a patient can tolerate. Peptide-mediated delivery of the drugs selectively to tumor tissue may alleviate this problem, because high concentrations of the drug could be attained within the tumor without affecting normal tissue. Moreover, blood vessels are easily accessible to intravenously administered therapy. Thus, by combining blood vessel destruction with the usual anti-tumor activities of a drug, a drug targeted to the vasculature of tumors can be expected to have increased efficacy and can be used at low enough doses to reduce the toxicity of chemotherapy.

One approach of targeted therapies is based on the specialization of the vasculature of individual organs at the molecular level. Endothelial cells lining blood vessels express tissue-specific markers. Binding of circulating chemotherapeutic agents delivered systemically to endothelial cell surface markers may induce localized cytotoxic effects. Targeting to tumor vasculature is promising as both primary tumor growth and the formation of metastasis depend on the establishment of new blood vessels from preexisting ones. Inhibition of angiogenesis and targeting of the tumor vasculature are highly effective in controlling tumor growth.

Targeting cancer therapy to endothelial cells is a rational approach because a clear correlation exists between proliferation of tumor vessels and tumor growth and malignancy. There are differences of cell membrane structures between tumor endothelial cells and normal endothelial cells which could be used for targeting of vectors. Moreover, tumor endothelial cells are accessible to vector vehicles in spite of the peculiarities of transvascular and interstitial blood flow in tumors. Based on the knowledge of the pharmacokinetics of macromolecules, it can be concluded that targeting tumor endothelial cells should have long blood residence time after intravascular application. A long blood residence time would allow a sufficient attachment to tumor endothelial cells.

Preferential homing of tumor cells and leukocytes to specific organs indicates that tissues carry unique marker molecules accessible to circulating cells. Organ-selective address molecules on endothelial surfaces for lymphocyte homing to various lymphoid organs and to tissues undergoing inflammation have been identified. Endothelial markers responsible for tumor homing to the lungs have also been identified.

A new approach to study organ-selective targeting based on in vivo screening of random peptide sequences has been reported. Peptides capable of mediating selective localization of phage to brain and kidney blood vessels were identified and showed up to 13-fold selectivity for these organs. It is possible to employ such targeting in a therapeutic setting (8-9). One peptide motif contained the sequence Arginine-Glycine-Asparagine embedded in a peptide structure that was shown to bind selectively to αᵥβ₃ and αᵥβ₅ integrins. A second peptide motif that accumulated in tumors contained the sequence Asparagine-Glycine-Arginine, which has been identified as a cell adhesion motif. Other peptides derived from the pathological vasculature have also been identified (10-12).

Based on the principle that tumor growth can be limited by restricting the blood supply, a wide variety of anti-angiogenic strategies have been developed, many of which involve systemic administration of macromolecules as bolus, repeated injections. Although the therapeutic index of some of these treatments may be high, less effort has been focused on sustained or targeted delivery of anti-angiogenic compounds. For example, nanoparticulate delivery systems are particularly suited to delivering a therapeutic, such as a drug, a chemotherapeutic or an immunotherapeutic, to an individual.

The prior art lacks methods of delivering a drug or other therapeutic over an extended time course. Specifically, the prior art is deficient in biocompatible, nanoparticulate formulations that are designed to retain and deliver anti-angiogenic peptides over an extended time course. The present invention fulfills this long-standing need and desire in the art.

### SUMMARY OF THE INVENTION

The present invention is directed to a nanoparticle or pharmaceutical composition thereof comprising a water-based core and a water-based corona surrounding the core. The core comprises at least one polyanionic polymer and a drug or therapeutic peptide which is crosslinked to or conjugated to a polymer. The water-based corona surrounding the core comprises at least one polycationic polymer and a targeting ligand which is cross-linked to or conjugated to a polymer. The nanoparticle further may comprise an inorganic salt and/or a bioluminescence agent or a contrast agent in the nanoparticle core and/or a cation in the corona.

The present invention is also directed to a related nanoparticle or pharmaceutical composition thereof comprising a water-based core and a water-based corona surrounding the core. The core comprises HV sodium alginate and cellulose sulfate and a drug or therapeutic peptide which is crosslinked to dextran polyaldehyde or conjugated to heparin sulfate. The water-based corona surrounding the core comprises spermine hydrochloride, poly(methylene-co-guanidine) hydrochloride, pluronic F-68 and calcium chloride. A targeting ligand is cross-linked to dextran polyaldehyde or conjugated to an activated polyethylene glycol. The nanoparticle further may comprise an inorganic salt and/or a bioluminescence agent or a contrast agent in the nanoparticle core and/or a cation in the corona.

The present invention is directed further to another related nanoparticle or pharmaceutical composition thereof comprising at least one low molecular weight polyanionic polymer in the water-based core and at least one low molecular weight polycationic polymer in the water-based corona. The core further comprises a drug or therapeutic peptide which is crosslinked to dextran polyaldehyde or conjugated to heparin sulfate or LMW sodium alginate or activated polyethylene glycol. The corona further comprises a targeting ligand is cross-linked to dextran polyaldehyde or conjugated to an activated polyethylene glycol. The nanoparticle may additionally comprise an inorganic salt and/or a bioluminescence agent or a contrast agent in the nanoparticle core and/or a cation in the corona.

The present invention also is directed to a method of delivering a drug or therapeutic peptide to a cell or tissue of interest in an individual. The nanoparticles comprising the drug or therapeutic peptide described herein are administered to the individual. The targeting ligand comprising the nanoparticles targets the nanoparticle to the cell or tissue of interest in the individual thereby delivering the drug or therapeutic protein thereto.

The present invention also is directed to a related method of imaging a cell or tissue of interest in an individual during delivery of a drug or therapeutic peptide thereto. The nanoparticles comprising the drug or therapeutic peptide and the bioluminescent/contrast agent described herein are administered to the individual. The nanoparticles are targeted to the cell or tissue via the targeting ligand comprising said nanoparticles while simultaneously the cell or tissue is imaged via the bioluminescence agent or contrast agent as the drug or therapeutic peptide is delivered.

The present invention is directed further to a method of producing a nanoparticle suitable for delivery of a drug or therapeutic protein to a cell or tissue of interest in an individual. The method comprises mixing at least one stream of a solution comprising components of the polyanionic core of the nanoparticle described herein with at least one stream of a solution comprising the components of the polycationic corona of this nanoparticle corona. The nanoparticles form a complex multipolymeric structure to crosslink or conjugate the drug or therapeutic protein comprising the core therewithin and to crosslink or conjugate the targeting ligand comprising the corona thereto. The complex structure of the nanoparticle is suitable to deliver the drug or therapeutic peptide to the cell or tissue of interest.

The method may further comprise one or more steps of adding a cation to the corona solution, adding an inorganic salt to the core solution or adding a bioluminescent agent or contrast agent to the core solution. Mixing of the streams may utilize simple flowing or laminar flowing. Additionally, the method may further comprise independent feedback monitoring in real time of a characteristic of the nanoparticle and/or of the process and optimizing the characteristic in real time. The method further may comprise washing the nanoparticles and, optionally, cryoprotecting and lyophilizing the nanoparticles.

Other and further aspects, features, and advantages of the present invention will be apparent from the following description of the presently preferred embodiments of the invention. These embodiments are given for the purpose of disclosure.

### DETAILED DESCRIPTION OF THE INVENTION

One embodiment of the present invention provides a nanoparticle or a pharmaceutical composition thereof comprising a water-based core having at least one polyanionic polymer; a drug or therapeutic peptide; and a polymer cross-linked to or conjugated to the drug or therapeutic peptide; and a water-based corona surrounding said core, comprising at least one polycationic polymer; a targeting ligand specific to a cell or tissue of interest; and a polymer cross-linked to or conjugated to said targeting moiety.

Further to this embodiment the nanoparticle may comprise a cation in the polycationic corona. An example of such cation is calcium chloride. Also, the nanoparticle may comprise a monovalent or a divalent inorganic salt in the polyanionic core. Examples of inorganic salts are sodium chloride and calcium chloride. Additionally, the nanoparticle may comprise a bioluminescent agent or a contrast agent in the polyanionic core. An example of a bioluminescent agent is luciferase. The contrast agent may be a macromolecular contrast agent or a dynamic contrast enhancing agent.

In an aspect of this embodiment the polyanionic polymer may be high viscosity sodium alginate (SA-HV), low molecular weight sodium alginate (LMW-SA), heparin sulfate, kappa carrageenan, low-esterified pectin (polygalacturonic acid), polyglutamic acid, carboxymethylcellulose, chondroitin sulfate-6, chondroitin sulfate-4, or collagen. Further to this aspect the polycationic polymer may be polyvinylamine, spermine hydrochloride, poly(methylene-co-guanidine) hydrochloride, protamine sulfate, polyethyleneimine, polyethyleneimine-ethoxylated, epichlorhydrin modified polyethyleneimine, quartenized polyamide, polydiallyldimethyl ammonium chloride-co-acrylamide, F-68-Pluronic copolymer, or chitosan.

In a related aspect the polyanionic polymers may be high viscosity sodium alginate, cellulose sulfate, the nanoparticle further comprising sodium chloride in the core; and the polycationic polymers are spermine hydrochloride, poly(methylene-co-guanidine) hydrochloride and F-68 Pluronic copolymer, the nanoparticle further comprising calcium chloride in the corona.

In another related aspect the polyanionic polymers may be high viscosity sodium alginate, cellulose sulfate, the nanoparticle further comprising heparin and calcium chloride in the core and the polycationic polymers are spermine hydrochloride, poly(methylene-co-guanidine) hydrochloride and F-68 Pluronic copolymer, the nanoparticle further comprising calcium chloride in the corona.

In another aspect of this embodiment the nanoparticle may comprise low molecular weight polyanionic polymers in the core and low molecular weight polycationic polymers in the corona. The LMW polyanionic polymers may be low molecular weight polyanionic polymers are LMW sodium alginate, LMW sodium hyaluronate, pentasodium tripolyphosphate, heparin sulfate or chondroitin sulfate. The LMW polycationic polymers may be LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, or LMW chitosan.

In a related aspect the LMW polyanionic polymers may be chondroitin-6-sulfate and heparin sulfate and the polycationic polymers are spermine hydrochloride, poly(methylene-co-guanidine) hydrochloride and F-68 Pluronic copolymer. Optionally, in this related aspect the polycationic polymers are spermine hydrochloride and F-68 Pluronic copolymer.

In another related aspect the LMW polyanionic polymers may be LMW sodium alginate and heparin sulfate and the LMW polycationic polymers may be spermine hydrochloride and poly(methylene-co-guanidine) hydrochloride where the nanoparticle further comprises calcium chloride in the corona. Optionally, in this related aspect the polyanionic polymer is LMW sodium alginate. In yet another related aspect the polyanionic polymers are LMW sodium alginate and heparin sulfate and said polycationic polymers are spermine hydrochloride, and LMW chitosan.

In all aspects of this embodiment the cross-linking or conjugating core polymer may be dextran polyaldehyde, LMW sodium alginate or heparin sulfate. The drug or therapeutic peptide may be a growth factor, a gene, angiostatin, endostatin, thrombospondin 1 or a peptide fragment thereof, or thrombospondin 2 or a peptide fragment thereof or a combination thereof. The cross-linking or conjugating corona polymer may be dextran polyaldehyde or activated polyethylene glycol. The targeting ligand may be TSP517, TSP521, apoE, a polysaccharide targeted to lectin or lectin targeted to a glycan.

In a related embodiment of the present invention there is provided nanoparticle or a pharmaceutical composition thereof comprising a water-based core having HV sodium alginate and cellulose sulfate; and a drug or therapeutic peptide crosslinked with dextran polyaldehyde where the core further comprises calcium chloride; or a drug or therapeutic peptide conjugated to heparin sulfate where the core further comprises sodium chloride; and a water-based corona surrounding said core, comprising spermine hydrochloride, poly(methylene-co-guanidine) hydrochloride and pluronic F-68; calcium chloride; and a targeting ligand conjugated to an activated polyethylene glycol or crosslinked to dextran polyaldehyde.

Further to this embodiment the nanoparticle may comprise a bioluminescent agent or contrast agent in said polyanionic core, as described supra. In all aspects of this embodiment, the drug or therapeutic peptide and the targeting ligand are as described supra.

In another embodiment of the present invention there is provided a nanoparticle or pharmaceutical composition thereof comprising a water-based core comprising at least one LMW polyanionic polymer; and a drug or therapeutic peptide crosslinked with dextran polyaldehyde; or a drug or therapeutic peptide conjugated to heparin sulfate or LMW sodium alginate; and a water-based corona surrounding said core, comprising at least one LMW polycationic polymer; and a targeting ligand conjugated to an activated polyethylene glycol or crosslinked to dextran polyaldehyde.

Further to this embodiment the nanoparticle may comprise a monovalent or a divalent salt in the core as described supra, in case LMW alginate is used in the corona solution. Also, the nanoparticle may comprise a cation in the corona as described supra. Additionally, the nanoparticle further may comprise a bioluminescent agent or contrast agent in said polyanionic core, as described supra. In all aspects of this embodiment, the drug or therapeutic peptide and the targeting ligand are as described supra. The LMW polyanionic polymers, the LMW polycationic polymers and the combinations thereof are as described supra.

In yet another embodiment of the present invention there is provided a method of delivering a drug or therapeutic peptide to a cell or tissue of interest in an individual, comprising administering nanoparticles described supra comprising the drug or therapeutic peptide to the individual; and targeting the nanoparticles to the cell or tissue via the targeting ligand comprising the nanoparticles, thereby delivering the drug or therapeutic protein to the cell or tissue in the individual.

Further to this embodiment the method comprises imaging the cell or tissue, where the nanoparticles comprise a bioluminescent agent or contrast agent in the polyanionic core. In all aspects of this embodiment the cell or tissue of interest may comprise tumor vasculature. Additionally, the bioluminescent agent or contrast agent and the nanoparticles comprising the polymers, the drug or therapeutic peptide, the cation and/or salt, and the targeting ligand are as described supra.

In a related embodiment of the present invention there is provided a method of imaging a cell or tissue of interest in an individual during delivery of a drug or therapeutic peptide thereto, comprising administering the nanoparticles comprising the bioluminescent agent or contrast agent and the drug or therapeutic peptide described supra to the individual; targeting the nanoparticles to the cell or tissue via the targeting ligand comprising said nanoparticles; and simultaneously imaging the cell or tissue via the bioluminescent agent or contrast agent comprising the core of the nanoparticles as the drug or therapeutic peptide is delivered, thereby imaging the cell or tissue of interest in the individual during delivery thereof. In this embodiment the cell or tissue of interest may comprise tumor vasculature.

In yet another embodiment of the present invention there is provided a method of producing a nanoparticle suitable for delivery of a drug or therapeutic protein to a cell or tissue of interest in an individual, comprising mixing at least one stream of a solution comprising components of the polyanionic core of the nanoparticle described supra with at least one stream of a solution comprising the components of the polycationic corona of the nanoparticle described supra; and forming nanoparticles having a complex multipolymeric structure to crosslink or conjugate the drug or therapeutic protein comprising the core therewithin and to crosslink or conjugate the targeting ligand comprising the corona thereto; wherein the complex structure of the nanoparticle is suitable to deliver the drug or therapeutic peptide to the cell or tissue of interest.

Further to this embodiment the method may comprise adding a cation to the corona solution. The cation may be present in the corona solution at a concentration of about 0.1 wt-% to about 1 wt-%. An example of a cation is calcium chloride. The method also may comprise adding a monovalent or divalent inorganic salt to the core solution. The salt may be present in the core solution at a concentration of about 0.5 wt-% to about 2 wt-%. Examples of an inorganic salt are sodium chloride and calcium chloride. Additionally, the method may comprise adding a bioluminescent agent or contrast agent to said core solution as described supra.

Further still to this embodiment the method may comprise independent feedback monitoring in real time of a characteristic of the nanoparticle or of the process or a combination thereof, where the characteristic comprises nanoparticle size, nanoparticle charge density, flow rates of streams, flow ratios, pH, salt content, or ethanol content; and optimizing the characteristic in real time. The methods may comprise further still washing the nanoparticles. Additionally, the nanoparticles may be cryoprotected and lyophilized.

In an aspect of this embodiment the mixing step may comprise laminar flowing of one or more streams each of the core solution and of the corona solution together in a continuous mode. Further to this aspect the laminar flow of at least one of the streams may be oscillated. A representative frequency of oscillation is about 5 Hz to about 200 Hz. Alternatively, the laminar flow of the streams may be pressurized. The streams may be pressurized independently up to about 200,000 psi. In another aspect the mixing step may comprise simple flowing of one stream of the core solution and one stream of the corona solution together in a batch mode and stirring the mixed solutions.

In all aspects the core polymers individually may be present in a concentration of about 0.01 wt-% to about 0.5 wt-%. The corona polymers individually may be present in a concentration of about 0.01 wt-% to about 5.0 wt-%. The drug may be present in a concentration of about 0.03 wt-% to about 0.4 wt-%. The targeting ligand is present in a concentration about 0.01 wt-% to about 5.0 wt-%. Additionally, the solutions may be mixed at a flow ratio of about 1:1 to about 1:12 polyanion:polycation polymers.

As used herein, the term “drug” shall refer to a chemical entity of varying molecular size, both small and large, either naturally occurring or synthetic, exhibiting a therapeutic effect in animals and humans. If not specifically referred to in context, drug may include any therapeutic protein, peptide, antigen or other biomolecules, such as growth factors and genes. A “small” drug may be incorporated within a nanoparticle comprising at least one corona polymer and at least one core polymer of low molecular weight, as defined infra.

As used herein, the term “microparticulate systems” shall refer to particles having diameter 1-2,000 μm such as microcapsules with a diameter of 100-500 μm or nanoparticles with a diameter range 1-1000 nm with small nanoparticles having a range preferable range of 10-300 nm. Collectively, these systems are denoted as drug delivery vehicles.

As used herein, the term “microcapsule” shall refer to microscopic, i.e., a few micrometers in size to few millimeters, solid object, having an essentially regular spherical shape, exhibiting a polymeric core and a polymeric shell. Usually, the polymeric core and the polymeric shell have opposite charges. For example, a polyanionic core may be covered by a polycationic shell or corona.

As used herein, the term “nanoparticle” shall refer to submicroscopic, i.e., less than 1 micrometer in size, solid object, essentially of regular or semi-regular shape.

The particles comprise a polymeric core and a polymeric shell that are opposite in charge.

For example, a polyanionic core may be covered by a polycationic shell or corona.

As used herein, the term “polymeric shell” or “corona” refers to the outer layer of the nanoparticle. This layer exerts a partial permeability control.

As used herein, the term “polymeric core” shall refer to the inner part of the nanoparticle, usually holding a drug to be delivered.

As used herein, the term “polycation” shall refer to a polycationic polymer.

As used herein, the term “polyanion” shall refer to a polyanionic polymer.

As used herein, the term “low molecular weight” shall refer to a weight less than about 60,000 daltons,

As used herein, the term “cryoprotecting” shall refer to substances used for suspension of particles, which upon their water removal in vacuum allow particles to remain in individual and nonaggregating states.

In the description of the present invention, the following abbreviations may be used: SA-HV, high viscosity sodium alginate; LMW-SA, low molecular weight sodium alginate; LMW-HY, low molecular weight sodium hyaluronate, HS, heparin sulfate; CS, cellulose sulfate; k-carr, kappa carrageenan; LE-PE, low-esterified pectin (polygalacturonic acid); PGA, polyglutamic acid; CMC, carboxymethylcellulose; ChS-6, chondroitin sulfate-6; ChS-4, chondroitin sulfate-4; F-68, Pluronic copolymer; PVA, polyvinylamine; LMW-PVA, low molecular weight polyvinylamine 3PP, pentasodium tripolyphosphate; PMCG, poly(methylene-co-guanidine) hydrochloride; SH, spermine hydrochloride; PS, protamine sulfate; PEI, polyethyleneimine; PEI-eth, polyethyleneimine-ethoxylated; PEI-EM, polyethyleneimine, epichlorhydrin modified; Q-PA, quartenized polyamide; pDADMAC-co-acrylamide, polydiallyldimethyl ammonium chloride-co-acrylamide; PBS, phosphate-buffered saline; ECM, extracellular matrix molecule.

The present invention provides a series of biocompatible, nanoparticulate formulations used as drug delivery vehicles that have been designed to retain and deliver peptides over an extended time course. These preparations permit modification to a desirable size, provide adequate mechanical strength and exhibit exceptional permeability and surface characteristics. The present invention provides nanoparticles that confer improved control of the permeability of the particles and the release rate of drug encapsulated therein.

Generally, these drug delivery vehicles may be formed from a variety of materials, including synthetic polymers and biopolymers, e.g., proteins and polysaccharides, and can be used as carriers for drugs and other biotechnology products, such as growth factors and genes or may be used to carry imaging agents. These drug delivery vehicles may comprise a core polymeric matrix in which a drug can be dispersed or dissolved. The core is surrounded by a polymeric shell.

A multicomponent vehicle is formed by polyelectrolyte complexation. In these systems, the multicomponent vehicle, e.g., nanoparticle, may comprise two polymers each in the core and in the corona. Alternatively, one polymer plus two oppositely charged polymers are used to assemble the vehicle or nanoparticle. For example, one polyanion and two polycations or two polyanions and one polycations are used.

Polyanionic polymer components may include HV-sodium alginate, LMW sodium alginate, heparin sulfate, cellulose sulfate, kappa carrageenan, low-esterified pectin (polygalacturonic acid), polyglutamic acid, carboxymethylcellulose, chondroitin sulfate-6, chondroitin sulfate-4, polyvinylamine or LMW polyvinylamine, and collagen. Representative polycationic polymer components include polyvinylamine, spermine hydrochloride, protamine sulfate, polyethyleneimine, polyethyleneimine-ethoxylated, polyethyleneimine, epichlorhydrin modified, quartenized polyamide, polydiallyldimethyl ammonium chloride-co-acrylamide, chitosan and Pluronic copolymer F-68.

For example, the nanoparticles may be synthesized from the polyanions high viscosity sodium alginate and cellulose sulfate and the polycations poly(methylene-co-guanidine) hydrochloride (PMCG) and spermine hydrochloride. Alternatively, the nanoparticles may comprise one or more polyanionic low molecular weight components, such as, but not limited to, low molecular weight sodium alginate, chondroitin sulfate or heparin sulfate. These LMW polyanionic polymers may form nanoparticles with one or more LMW polycationic polymers, such as, but not limited to, spermine hydrochloride, chitosan, poly(methylene-co-guanidine) hydrochloride and F-68.

Additionally, a nanoparticle having a polycationic corona may comprise an inorganic salt, such as calcium chloride. Also a nanoparticle with a polyanionic core may comprise a monovalent or bivalent inorganic salt, such as sodium chloride, calcium chloride, or sodium sulfate. This increases the stability of the nanoparticles and results in, inter alia, increased entrapment efficiency for a more efficacious delivery of a biomolecule, such as a drug or imaging agent, contained within the core of the particle.

Drugs comprising the nanoparticulate complexes exhibiting charged character become an integral part of the particle. For example, an anionic antigen and polyanionic core polymers become an integral part of the complex formed with polycationic corona polymers. A nanoparticle having a polycationic core may incorporate a cationic drug. Non-charged small drugs are conveniently attached to larger molecules, preferably charged polymers. The nanoparticles may comprise a protein or drug which is, although not limited to, an anti-angiogenic factor. Representative anti-angiogenic factors include angiostatin, endostatin, thrombospondins 1 and 2 and their fragments, i.e., peptides.

To slow the release rate of the drug carried by the nanoparticles, the drug or peptide molecule can be covalently conjugated through a persistent chemical bond or cross-linked through a dissociable Schiff-base bond with at least one core polymer in the nanoparticle. Physiological reaction conditions are selected that induce a dissociable Schiff-base complex that provides slow drug release. The drug or peptide molecule may include various proteins, growth factors, antigens, or genes in addition to synthetic or naturally occurring chemicals.

In the formation of persistent covalent bond, a water-insoluble drug can be conjugated to a water-soluble polymer to solubilize the drug. Alternatively, one can form a conjugate between a water-soluble polymer and water-soluble drug. The conjugate of drug and polymer is then incorporated into a drug carrier of the present invention, including nanoparticles and microparticles. The entire conjugate of drug and soluble polymer is released from the particles by diffusion or by enzymatic degradation of the delivery vehicle.

Additionally, a smaller low molecular weight nanoparticle-drug or peptide complex may be used for delivery thereof. For example, a corona of polycationic or polyanionic formed from low molecular weight polymers and a core of polyanionic or polycationic polymers formed from low molecular weight polymers may contain a drug or peptide molecule of interest crosslinked or conjugated to a small molecular weight polymer, such as dextran polyaldehyde, LMW sodium alginate or heparin sulfate.

Furthermore, the invention includes polymeric complexes in which a gelling polymer and/or a polymer for permeability control which normally are charged polymers of opposite charge to the drug molecules are used to slow the diffusion rate of the charged drugs from the nanoparticles. The gelling polymer is typically a core polymer, such as alginate. The polymer for permeability control is typically a corona (shell) polymer, such as poly(methylene-co-guanidine) hydrochloride or spermine hydrochloride.

The corona periphery may be modified further by including a targeting ligand for specific delivery to a cell or tissue site. Preferably, the nanoparticles are targeted to an organ or tissue by a ligand, such as TSP517, TSP521, apoE, polysaccharide capable of targeting to lectin molecule on cell surface or lectin capable of targeting to glycan motif on cell surface. For example, a conjugate of a ligand, for example the peptide TSP-517, with activated PEG may be used to target the nanoparticle to the site of interest. Targeting to tumor vasculature can be mediated by peptide targeting or by glycan or lectin-based ligands attached to the periphery of the nanoparticles.

In addition to selective targeting of endothelial cells, the nanoparticles also may comprise a noninvasive imaging agent by incorporating a bioluminescence agent, such as luciferase, and/or magnetic resonance imaging constrast agent, such as, a macromolecular contrast agent or dynamic contrast enhanced agent. An example of a contrast agent is, but not limited to, polymeric gadolinium contrast agent. As such, the present invention also provides methods of using the claimed nanoparticles to deliver a drug to a targeted tissue, such as tumor vasculature. When the nanoparticles further incorporate a bioluminescence agent or a contrast agent, simultaneous drug delivery and imaging of the targeted tissue can be performed.

Thus, pharmaceutical compositions may be prepared using a drug encapsulated in the delivery vehicle of the present invention. In such a case, the pharmaceutical composition may comprise a drug, e.g., anti-vascularization agent, and a biologically acceptable matrix. Suitable polymeric forms include microcapsules, microparticles, films, polymeric coatings, and nanoparticles.

Nanoparticles are particularly useful in the practice of the invention. Prior to use the nanoparticles may be cryoprotected or lyophilized to extend the therapeutic life of the nanoparticle. Cryoprotecting the nanoparticles, with concomitant stabilization, is provided by means of lyophilization. The washed particles are suspended in a cryoprotective solution and lyophilization of the suspension is performed in a suitable lyophilization apparatus. Such cryoprotective solutions may include glycerol, trehalose, sucrose, PEG, PPG, PVP, block polymers of polyoxyethylene and polyoxypropylene, water soluble derivatized celluloses and some other agents at a concentration of 1 wt-% to 10 wt-%.

Because of their small size and suitability for use in injectable formulations. These nanoparticles can be administered locally or systemically. For example, a pharmaceutical composition comprising the nanoparticles of the instant invention may be administered orally, intravenously, nasally, rectally or vaginally, through inhalation to the lung, and by injection into muscle or skin or underneath the skin. Additionally, those polyelectrolyte complexes with a polyanionic or polyanionic/salt core that are administered intravenously demonstrate a greater encapsulation efficiency of the drug and stability in sera.

A person having ordinary skill in this art would readily be able to determine, without undue experimentation, the appropriate concentrations of the biotechnology products, such as drugs or imagining agents, amounts and routes of administration of the drug delivery vehicle of the present invention to deliver an efficacious dosage of drug or other agent over time. Furthermore, one of ordinary skill in the art may determine treatment regimens and appropriate dosage using the nanoparticles of the present invention without undue experimentation. An appropriate dosage depends on the subject's health, the progression or remission of the disease, the route of administration and the nanoparticle used.

The nanoparticles of the present invention may be prepared by providing a stream of uniformly-sized drops of a charged polymer solution in which the particle size of the drops is submicron or at most only a few microns, collecting these droplets in a stirred reactor provided with a polymeric solution of opposite charge, and reacting the droplets and the solution to form the particles. When the drops of polymer are polyanionic and the receiving polymer solution is cationic, the particles have a polyanionic core and a shell or corona of a polyanionic/polycationic complex. The periphery of the particle has an excess positive charge. Conversely, drops of a stream of cationic solution can be collected in a polyanionic solution. These particles have polycationic core and shell of a polycationic/polyanionic complex with an excess of negative charge on the particle periphery.

Alternatively, the nanoparticles may be prepared utilizing a mixing device, e.g., microfabricated mixing device, of complex geometry. Flow rates may be continuous or may be pulsed. The oscillatory flow of at least one fluid provides increased fluid flow for mixing and improved processing. Thus, the process is scaled-up.

Mixing devices that use multiple, reactant fluid streams with very high mixing energy density and enhanced mixing intimacy of reactants provide fast and controlled reaction chemistry not available from conventional batch reaction technology. U.S. Pat. No. 6,221,332 provides a means to develop and manufacture nanomaterials in a process controllable to the “molecular level of mixing. Generally, the microfabricated design, in that the system may be scaled-up, provides a much higher throughput and, unlike batch processes, can be operated continuously.

The mixing device may be coupled to a device, such as an autotitrator, which can measure the size or charge density of nanoparticles, in real time, within the output of the mixing device, providing for feedback and correction of the chemistry of the reacting streams, in terms of ratio of flow of individual streams, pH of the streams, salt content of the streams and, alternatively, ethanol content, as a de-solvating agent, within one of the streams, in order to control the final output of the process

The individual components of the core polyanionic solution of polymers, including crosslinking or conjugating polymers, may have concentrations of 0.01 wt-% to 0.5 wt-%. In a more preferred composition each component of the core polyanionic solution is at a concentration of 0.03 wt-% to 0.2 wt-%. The drug may be present in the core solution at a concentration of about 0.05 wt-% to about 0.4 wt-%. Calcium chloride and sodium chloride individually may be at a concentration of 0.05 wt-% to 0.2 wt-%.

In addition, the individual components of the corona cationic solution are at a concentration of 0.01 wt-% to 0.5 wt-%. Pluronic F-68 is at a concentration of 0.1 wt-% to 5 wt-%. The targeting ligand may be present in the corona solution at a concentration of 0.01 wt-% to 0.5 wt-%. Calcium chloride may be present at a concentration of 0.05 wt-% to 0.2 wt-%.

The following examples are given to illustrate various embodiments of the invention and are not meant to limit the present invention in any fashion.

### EXAMPLE 1

### Anti-Angiogenic Factor-Loaded Nanoparticle

Particles were generated using a droplet-forming core polyanionic solution of 0.05 wt-% HV sodium alginate (SA-HV), 0.05 wt-% cellulose sulfate (CS) in water, 0.05 wt-% TSP-1 in water, also containing 2 wt-% NaCl (Sigma; St. Louis, Mo.), and a corona-forming polycationic solution of 0.05 wt-% SH, 0.05 wt-% poly(methylene-co-guanidine) hydrochloride (PMGH), 0.05 wt-% calcium chloride, and 1 wt-% F-68 in water. Typical ranges of concentrations for these polymers are 0.03-0.06 wt % for HV-SA, 0.03-0.06 wt % for cellulose sulfate, 0.03-0.06 wt % for SH, 0.035-0.55 wt % for PMCG, 0.05-2 wt % for sodium or calcium chloride and 0.01-5 wt-% for F-68.

The polymers were high viscosity sodium alginate (SA-HV) from Kelco/Merck (San Diego, Calif.) of average molecular weight 46,000; cellulose sulfate, sodium salt (CS) from Janssen Chimica (Geel, Belgium), average molecular weight 1,200,000; poly(methylene-co-guanidine) hydrochloride (PMCG) from Scientific Polymer Products, Inc. (Ontario, N.Y.), with average molecular weight 5,000; spermine hydrochloride (SH) from Sigma, molecular weight 348.2. TSP-1 (Sigma) is a matricellular anti-angiogenic factor, thrombospondin-1, derived from platelets, average molecular weight 83,000. Pluronic P-68 (Sigma) of average MW 5,400, is a water-soluble nonionic block polymer composed of polyoxyethylene and polyoxypropylene segments.

The particles were instantly formed and were allowed to react for 1 hour. The encapsulation efficiency was 5%. The nanoparticle size and charge was evaluated in the reaction mixture by centrifugation at 15,000 g. The average size was 230 nm and the average charge 15.2 mV. The particles were resuspended with different buffers at neutral pH 7, pH 1.85 and pH 8 and TSP-1 release was measured by a colorimetric method (Bradford). The product is stable in water, in neutral buffers, in 0.9 wt-% saline and in animal sera. These nanoparticles also were tested in the presence of 0-2 wt-% NaCl or 0-2% calcium chloride added into the droplet-forming solution. The amount of entrapped TSP-1, i.e., encapsulation efficiency, increased dramatically for both sodium and calcium chlorides to about 50%.

### EXAMPLE 2

### Anti-Angiogenic Factor-Loaded Crosslinked Nanoparticle

These particles were generated using the same solutions as in Example 1, except the droplet forming solution contained additional polymer, DPA and 1 wt-% calcium chloride instead of sodium chloride. DPA is dextran polyaldehyde from CarboMer (Westborough, Mass.) with an average molecular weight of 40,000. In addition, the core solution contained ¹²⁵I-labeled TSP-1, instead of nonlabeled TSP-1. The TSP-1 labeling was done by means of a labeling kit (Pierce).

The particles were instantaneously formed, allowed to react for 1-hour and their size and charge evaluated in the reaction mixture. The average size was 250 nm and the average charge 15.5 mV. The particles were separated by centrifugation and were incubated for 30 min in a HEPES buffer at pH 8.0 to perform the crosslinking reaction between the polymer constituents and TSP-1.

The DPA/TSP-1 mass ratio was: 0 (no crosslinking), 0.01, 0.05 and 0.1. The higher the ratio of DPA/TSP-1, the slower the release rate of the drug. The Schiff-base product between the anionic groups of TSP and aldehyde group of DPA allowed an adjustment of release via ion exchange. The adjustment is made via the amount of Schiff-base product introduced and the degree of dissociation of this covalent bond, depending on in vitro and in vivo conditions. The release rate was adjusted to any value between 3 and 10% per day, amounting to approximately 30 to 10 days of cumulative delivery time.

The tracer quantity was assayed using a gamma counter and permeability assessed via an efflux method (13). Particles with different levela of crosslinking have different permeability and drug release rates. More crosslinked nanoparticles would have lower drug release rates. Similar results were obtained when the anionic solution was pre-incubated first at pH 8.0 for 30 min and the particles formed after incubation of the solution.

Another set of nanoparticles was made in a similar fashion, except the droplet-forming solution contained different amounts of heparin sulfate (Sigma). The ratios tested were 20:1, 10:1, 2:1, 1:1 and 1:2 of TSP-1:heparin sulfate. Release rates were slowed down to 0.5 to 3% per day in presence of heparin as compared to 50% per day for non-crosslinked nanoparticles. Thus, the drug release rate of the nanoparticles can be adjusted over a wide range to suit different therapeutic needs. The drug release rate can be lowered by increasing the extent of cross-linking or conjugation.

### EXAMPLE 3

### Nanoparticles With Covalent Conjugate of Peptide Molecule And Polymer

A drug peptide or targeting peptide may be conjugated to a polymer to reduce the rate of release of a peptide. TSP-517 is a peptide of 1642 Da derived from the thrombospondin molecule, and has the amino acid sequence KRAKQAGWSHWAA (SEQ ID NO. 1). This peptide has a heparin-binding motif and is capable of binding to sites on the tumor vasculature. TSP-517 peptide was synthesized by solid-state chemistry in-house (14).

To incorporate TSP-517 into nanoparticles, the peptide was conjugated to an activated polyethylene glycol, mPEG-SPA with average molecular weight 20,000 (Shearwater Polymers, Huntsville, Ala.). Conjugate was separated from free peptide by dialysis and then purified by affinity chromatography on heparin-Sepharose. The highest yields of conjugate were obtained with a 2:1 ratio of PEG to peptide. Although gradient elution yielded three overlapping peaks in the bound fraction, each showed an identical mobility by SDS-PAGE consistent with a 1:1 molar ratio. The conjugate was incorporated into the nanoparticles during their fabrication as in Example 2.

A separate batch of nanoparticles was prepared in the presence of a small amount of adenoviral luciferase plasmid in the core polymer solution. The adenoviral construct containing luciferase gene was prepared as follows. 293 adenovirus transformed human embryo kidney cells were grown in Dulbecco's modified Eagle's medium (DMEM) with 10% fetal calf serum (FCS) supplemented with 2 mM L-glutamine. The XbaI/SmaI DNA fragment containing an internal ribosome entry site (IRES) and GFP (Green Fluorescent Protein) isolated from pIRES-GFP (Clontech, Palo Alto, Calif.) and another XbaI/XhoI luciferase DNA fragment cut from pGL-Basic (Promega) were separately subcloned into pShuttle-CMV vector (Quantum Biotechnologies, Montreal, Canada).

The resulting plasmid was co-transformed into BJ5381 cells with pAdEasy-1 adenoviral DNA plasmid that was E1 and E3 deleted and replication-deficient. The recombinant adenoviral construct was linearized with Pac I and transfected into 293 cells in which E1 functions can be complemented in order to produce viral particles. To achieve a large adenovirus preparation, Ad-luc-IRES-GFP was amplified in 293 cells cultured in cell factories (Nalgene Nunc), purified by cesium chloride centrifugation, desalted with PD-10 column (Amersham Pharmacia Biotech, Uppsala, Sweden) and stored at −80° C. The viral titer was determined with the cytopathic effect assay (TCDI ₅₀) on 293 cells and calculation was done according to the protocol of Quantum Biotechnologies.

To evaluate biodistribution of targeted nanoparticles, mice that had been implanted with polyvinylalcohol sponges as model wounds representing neovasculature of tumor (15) were administered either free adenoviral luciferase (Ad-luc) plasmid or conjugated TSP-517/PEG nanoparticles containing the same amount of adenovirus by tail vein injection. Luciferase was used for nanoparticle visualization by means of a bioluminiscence CCD camera. Luciferase activity was evaluated 4 days after injection.

Free virus localized predominantly to the liver with minor distribution to lung and spleen and in sponge granulation tissue. In contrast, luciferase expression was more widely distributed in mice injected with TSP-PEG nanoparticles. The lung was a significant reservoir and significant luciferase activity was detected in sponge homogenates. The targeted nanoparticles were much less partitioned into the reticular endothelial system (RES) and more into proliferating endothelial cells and pericytes.

Further optimization of conjugate loading by means of multivalent PEG's can further modify the distribution of nanoparticles in favor of neovascular sites. In addition to TSP517, other targeting peptides such as TSP521, ApoE494 peptide which is a monomeric version of ApoE peptide (16) and Ruoslahti's homing peptide (17) could be used. All these peptides have a capability to bind a corresponding motif on the endothelial cell lining of tumor blood vessels.

### EXAMPLE 4

### Biocompatibility Test and Supression of Vascularization

Crosslinked nanoparticles loaded with 0.1-10 μg/batch (0.1 ml of the final product) TSP-1 were prepared as described in Example 2. Nanoparticles loaded with a control angiogenic substance bFGF (10 μg/0.1 ml) were also prepared. A 1:1 mixture of TSP-loaded and bFGF-loaded nanoparticles and bFGF-loaded nanoparticles alone, as a control, were placed subcutaneously or intraperitoneally into Sprague-Dawley rats, each receiving 0.2 ml, and evaluated at days 8, 48 and 96.

Visual observations, backed by histology (inflammatory reactions, degree of fibrosis and development of granulation tissue with capillaries), were supplemented by detection of a specific vascularization marker using an antibody against Factor VIII, i.e., von Willebrand factor, a specific lectin I/B4 or anti-collagen type IV antibody (Vector Laboratories, Burlingame, Calif.) (18). The data collected clearly indicated that normal angiogenesis due to wound healing is suppressed by means of immobilized anti-angiogenic factor applied in the form of nanoparticles for certain ratio of TSP-1/bFGF.

Biocompatibility of the empty nanoparticles, with no TSP or bFGF, prepared as above was determined in the subcutaneous and intraperitoneal sites in rats. Histology and histochemistry of all implants included standard techniques (19-20). No adverse reactions were noted.

### EXAMPLE 5

### Animals Survival Studies

The nanoparticle delivery vehicle similar to that in Example 2 was assembled. It contained core-loaded TSP-1 and corona loaded TSP-521 peptide-PEG conjugate. A slow-release of the core drug peptide is more important for achieving more meaningful therapeutic effects. Thus, to allow for controlled release of the core-loaded peptide, the release rate was adjusted by means of DPA crosslinking. Such crosslinking partially immobilized the corona-entrapped targeting peptide as well.

The following three doses of TSP-1 were applied: 150 μg, 80 μg and 10 μg. The cross-linked peptide was designed for slow-delivery over 10 days period. Moreover, the amount of targeting peptide was adjusted to allow for optimal capture of the nanoparticles in the tumor vasculature. An optimal amount of targeting is that allowing for retention but not dislocation of particle within the tumor area.

Particles also can be generated using a droplet-forming polyanionic solution composed of 0.05 wt-% HV sodium alginate (SA-HV), 0.05 wt-% cellulose sulfate in water, 0.05 wt-% TSP-1 in water and 2 wt-% NaCl (Sigma), and a corona-forming polycationic solution composed of 0.05 wt-% SH, 0.05 wt-% poly(methylene-co-guanidine) hydrochloride, 0.05 wt-% calcium chloride, 1 wt-% F-68 and 0.01 wt-% TSP521 peptide conjugate with mPEG-SPA as prepared in Example 3. The core-loaded TSP-1 functions as a therapeutic anti-angiogenic peptide, whereas the corona associated TSP521 is a targeting peptide.

For animal studies, a total of 20 tumor bearing mice were used, half of which received injection of core-loaded TSP-1 nanoparticles with the corona-loaded TSP521-conjugate. The other half, as controls, received nanoparticles loaded with a corona-attached control scrambled, inactive peptide conjugated to mPEG-SPA. Subcutaneous tumors were produced by local injection of 5×10⁵ 4T1 cells, while liver tumors were produced by injection into the portal vein. Lung metastases occurred spontaneously. In a separate study, tumor response rates were determined for 8 weeks and compared to controls. As a primary measure of the effect of the anti-angiogenic therapy, the animal survival rate was used as the first assessment (Table 1).

TABLE 1
Treatment of Tumors By Targeted Nanoparticles

| | Days (d) | | | | | |
|---|---|---|---|---|---|---|
| Survival (%) | d 14 | d 21 | d 28 | d 35 | d 42 | d 56 |
| Test Animals | 100 | 100 | 100 | 90 | 80 | 40 |
| Control Animals | 100 | 100 | 60 | 20 | 0 | 0 |

### EXAMPLE 6

### Noninvasive Imaging Using Luciferase Bioluminescence and Magnetic Resonance Imaging With Gadolinium Contrast

The nanoparticle delivery vehicle similar to that in Example 3 was assembled. It contained core-loaded TSP-1 and corona loaded TSP-521 peptide-PEG conjugate. In addition, the core polymer solution also contained luciferase (Sigma). Nanoparticles were injected into mice bearing tumor via the tail vein and tissue distribution was visualized with an iCCD at 1, 6, 24, 48, and 72 h after injection. The TSP521 peptide will allow trafficking of nanoparticles to the tumor, whereas luciferase activity will allow visualization of the nanoparticles.

In a similar experiment, nanoparticles were prepared as above, except the core solution also contained the macromolecular gadolinium contrast agent Magnevist (Berlex Laboratories). Animals with tumors were imaged on the 4.7 T Animal Imager under general gas anesthesia to reduce motion. Animals were placed in a holder in a linearly polarized circular coil and imaged with two different pulse sequences. The first pulse sequence yielded T2* sensitivity and was used to estimate vascular dynamics. Dynamic contrast imaging uses a pulse sequence that detects the passage of the agent through the tissues (21).

The 4.7 T system can acquire data at about 1 image/second, which is sufficient to characterize the magnetic susceptibility changes during passage of a macromolecular gadopentetate dimeglumine contrast agent. This reagent has a blood half-life of 36 hrs in rats. The resulting images, when processed, can provide blood volume and temporal characteristics of the capillary beds of interest.

Once the contrast agent has equilibrated, a T2-weighted, multi-slice, spin echo pulse sequence (TR=2000, TE=25) yields enhancement for visualization of the tumor volume. Multiple slices were processed after collection by selecting regions-of-interest on each image to produce an estimate of size and volume of the tumor. Macromolecular contrast agents quantitatively assay microvascular hyperpermeability and produce an increased signal-to-noise ratio. On the other hand, dynamic contrast enhanced agent, a low molecular gadolinium, quickly equilibrates between blood and the extracellular space and doesn't provide a long-term signal related to microvascular density. Combined use of nanoparticles for targeting, therapy and imaging was thus demonstrated.

### EXAMPLE 7

### Targeting With Help of a Lectin Polysaccharide

A tetrasaccharide (A-tetra) specific for Galectin-3 was obtained from Biocarb. Its composition is as follows: GalNAc alpha1-3Gal beta1-4Glc (−2 Fuc alpha1) (22). The preparation of Dex/A-tetra conjugate was carried out according to the following procedure. Dextran (1000 mg, 4.5 mmol in sugar unit, molecular weight 4.2×10⁴, Sigma) was dissolved in dimethyl sulfoxide (DMSO, Sigma). 4-Nitrophenylchloroformate (650 mg, 3.2 mmol, Sigma) and 4-(dimethylamino)pyridine (DMAP, Sigma) (350 mg, 2.8 mmol) were added to the ice-cooled solution. The reaction mixture was stirred at 0° C. for 4 h and then re-precipitated by acetone/diethyl ether/ethanol (1:1:2, v:v:v) to give Detran-activated ester. The activated ester was dissolved in DMSO, and then A-tetra was added to the solution.

The mixture was stirred at room temperature for 36 h. After evaporation, the residue was dissolved in DMF and subjected to gel-filtration chromatography (Sephadex LH-20; column, o.d. 40×550 mm; eluent, DMF) to give Dex/A-tetra conjugate. The degree of introduction of Gal units per sugar unit was estimated to be 2.9 mol % from the N/C ratio of the elemental analysis. Yield: 520 mg.

A control conjugate having no galactose residues was also synthesized; saccharose was used instead. These conjugates were used for the investigations of interactions with lectin (Galectin-3). The interactions of dextran derivatives with Galectin-3 lectin were evaluated by calorimetric titration (22). Results of the interaction between the lectin and dextran derivatives showed high apparent affinity constants for active conjugate.

The nanoparticle delivery vehicle similar to that in Example 2 was assembled. It contained core-loaded Doxorubicin-polymer conjugate and corona loaded Dex/Tetra-A conjugate. The processes of targeting can be controlled by the absolute amounts of Dex/Tetra-A corona-loaded material. Nanoparticles exhibited a high affinity to a squamous tumor cell tissue section and to a head and neck cancer cell line as detected histochemically or by means of fluorescence. A fluorescing polymer core-entrapped in the nanoparticles was used to simplify the observation (23). In a similar way, targeting based on lectin instead of glycan was also tested. A lectin Sambus nigra agglutinin (SNA) (Vector Laboratories, Burlingame, Calif.) was incorporated into the nanoparticle corona by entrapment with a goal of targeting it to appropriate cell-based receptor, i.e., sugar-based, on the cell surface of gastrointestinal tract, e.g., CaCo cells.

### EXAMPLE 8

### Nanoparticulate Composition With Low Molecular Weight Chemistry Chondroitin-6-Sulfate and Heparin Sulfate in the Anionic Core

Particles were generated using a droplet-forming polyanionic solution composed of 0.1 wt-% chondroitin-6-sulfate (ChS), 0.1 wt-% heparin sulfate (HS) in water, and a corona-forming polycationic solution composed of 0.1 wt-% spermine hydrochloride (SP), 0.1 wt-% PMCG hydrochloride, and 1 wt-% F-68 in water. Typical range for ChS 0.05-0.15 wt %, for HS 0.05-0.15 wt %, for SP 0.05-0.15 wt %, for PMCG 0.05-0.15 wt %, and for F-680.05-5 wt-%. The anionic solution contained additional polymer, ovalbumin, as a representative protein drug. The amount was about 0.05-4 wt-%. The pH of the polyanionic solution was adjusted within the pH 8.3-11 range by means of diluted sodium hydroxide.

The polymers were low molecular weight chondroitin-6 sulfate (Sigma, St. Louis, Mo.) of average molecular weight 15,000; heparin sulfate, sodium salt (HS) from Sigma (St. Louis, Mo.), with average molecular weight 7,000; poly(methylene-co-guanidine) hydrochloride (PMCG) from Scientific Polymer Products, Inc. (Ontario, N.Y.), with average molecular weight 5,000; spermine hydrochloride (SH) from Sigma, with molecular weight 348.2; and Pluronic P-68, from Sigma, with average molecular weight 5,400.

The particles were instantaneously formed by bringing two polymeric streams, in the ratio 1:8, polynion/polycation, together in a stirred vessel; then, they were allowed to react for 1 hour. The entrapment efficiency was 55% for pH 8.3 of the anionic solution. The entrapment efficiency dramatically increased to 80% when the pH of the anionic solution was increased from pH 8.3 to 11 and tested in steps.

The nanoparticle size and charge was evaluated in the reaction mixture and after the centrifugation at 15,000 g by means of Malvern instrument (ZetaSizer, Malvern, UK) and by transmission electron microscopy. The average size was 85 nm and the average charge 18.8 mV. The product is stable in water, neutral buffers, in 0.9 wt-% saline and in animal sera. Similar results were obtained if only one polycation was used, for example when PMCG was omitted. These nanoparticles can be derivatized for targeting as exemplified in Example 3 and 7, and for slow-release as in Example 2.

### LMW-Sodium Alginate and Heparin Sulfate in the Anionic Core

Particles were generated using a droplet-forming polyanionic solution comprising 0.05 wt-% low molecular weight sodim alginate (LMW-SA), 0.05 wt-% heparin sulfate (HS) in water and a corona-forming polycationic solution comprising 0.05 wt-% SH, 0.05 wt-% PMCG hydrochloride, 0.1 wt-% calcium chloride and 1.0 wt-% F-68 in water. A typical range for each of LMW-SA, HS and SH is about 0.03-0.06 wt-%, for PMCG is about 0.035-0.55 wt-%, for calcium chloride is about 0.01-1 wt %, and for F-68 is about 0.01-5 wt-%. The anionic solution contained additional polymer, ovalbumin, as a representative protein drug. The amount was about 0.05-4 wt-%.

The polymers were LMW-SA (FMC BioPolymers, Philadelphia, Pa.) with an average molecular weight of 37,000; heparin sulfate, sodium salt with an average molecular weight of 7,000, spermine hydrochloride with an average molecular weight of 348.2, and Pluronic P-68 with an average molecular weight of 5,400, all from Sigma (St Louis, Mo.); and poly(methylene-co-guanidine)hydrochloride with an average molecular weight of 5,000 (Scientific Polymer Products, Inc., Ontario, N.Y.). P-68 is a water soluble nonionic block polymer composed of polyoxyethylene and polyoxypropylene segments.

The particles were formed instantaneously by bringing two polymeric streams, at a ratio of 1:8 polyanion/polycation, together in a stirred vessel and allowed to react for 1 hour. The entrapment efficiency of ovalbumin was 50%. The nanoparticle size and charge was evaluated in the reaction mixture and after centrifugation at 15,000 g by means of a Malvern instrument (ZetaSizer, Malvern, UK) and by transmission electron microscopy. The average size was about 80 nm and the average charge was 20.2 mV. The product is stable in water, neutral buffers, in 0.9 wt-% saline and in animal sera. Additionally, similar results were obtained for nanoparticles formed with only one polyanion, for example, only with LMW-SA with heparin omitted. These nanoparticles can be derivatized for targeting as exemplified in Example 3 and 7, and for slow-release as in Example 2.

### EXAMPLE 9

### Preparation of Nanoparticles by Mixing in a Microfabricated Device

Particles may be generated using the chemistry in Example 8 with a microfabricated mixing device. The device geometry was similar to that described by Stremler (24) except that it was fitted with two inlets. The size of channels was about 5×5 mm and it was made from plexiglass (PMMA) polymer. The device allows for laminar mixing in a 3-dimensional channel geometry. The ratio of flow rats was kept 1:8 polyanion/polycation and actual flow rates were 5 and 40 ml/min provided by peristaltic pumps. Once the device reaches a steady state over a few minutes, samples were collected and evaluated in terms of optical density (320 nm), size and charge.

Additional runs were made with ovalbumin entrapment, with 0.2-0.5 wt-% of OVA was incorporated into the anionic solution. Entrapment efficiencies were in the range of 50-60%. The microfabricated device provided a much higher throughput rate, 100 mg dry weight/min as compared to 3 mg dry weight/min of batch processing in Example 8. In addition, the microfabricated design allowed for continuous operation.

### EXAMPLE 10

### Nanoparticle Preparation Via a Microfabricated Device Plus Fluid Pulsing

Nanoparticles were prepared as in Example 8 except that one or two fluid streams was delivered in a pulsating, i.e., oscillatory, flow regime. For pulsing, a special solenoid valve connected to a frequency power source providing 5-100 Hz frequencies (Precision Dispensing, Bay Village, Ohio) was employed. This set-up allowed independent control of flow rate, as in Example 9, as well as control of the pulsing frequency, i.e., degree of mixing. Mass transfer and mixing is enhanced dramatically with one or two fluids operating in an oscillatory mode (25-26).

The outcome was a more uniform size distribution, evidencing the role of micromixing in the particle assembly process and better process control. With frequencies of 10-30 Hz, the entrapment efficiencies were somewhat higher as compared to Example 9 when one fluid, the cationic, was oscillated. A useful range of frequencies is between 5 and 200 Hz. Similar results were obtained for both fluids in an oscillatory mode. The oscillatory flow, of at least one fluid, allows for increased fluid flow for mixing and improved processing, as evidenced by dye tracer studies. Thus, higher flow rates were tested, ranging from 10-20 ml/min on the anionic side to 80-160 ml/min on the cationic side. The process scale-up is accomplished.

### EXAMPLE 11

### Process Optimization With Help of Control Feedback

Nanoparticles were prepared essentially as in Example 10, with flow rates ratio of 1:8 and individual rates of 10 ml/min for anionic streams and 80 ml/min for cationic streams. To accomplish the feedback on the process design and optimization, a Malvern autotitrator was connected to the microfabricated mixing device outlet. Specifically, the ratio of two polymeric streams, anionic/cationic, was changed in steps from 1:6 to 1:12 and the charge density of the nanoparticles was measured on-line. The charge density changed from 15.1 mV to 35.6 mV. Charge density is important for the passive biodistribution of the product among different organs, following intravenous injection of the nanoparticles. Similarly, a minimum nanoparticle size of about 60 nm ±5 nm was found following the optimization of the fluid rate ratio.

### EXAMPLE 12

### Molecular Mixing by Means of High-Pressure Microfluidics Device

Microfluidics Inc. (Newton, Mass.) offers a line of liquid processing equipment that is suited for production of micro- and nanoparticles that benefit from high mixing energies. A new Two Stream Mixer Reactor (TSMR) prototype was used. In most conventional chemical reactors, inadequate mixing and mass-transfer rates limit the value and performance of a fast chemical reaction. As a result, product yields are low and unwanted by-products are produced. The Microfluidizer technology utilizes pressurizing liquids and converting the pressurized energy to intense mixing in a mixing chamber, achieving residence times of a few tens of microseconds to a few hundred milliseconds.

Two plunger pumps were employed for independent pressurizing of the individual reactant streams to a high level, up to 200,000 psi. The prototype was adjusted to accommodate different flow rates of the two reacting streams, 10 ml/min and 80 ml/min, respectively, that is for anionic and cationic streams. Results demonstrated that the size distribution of nanoparticles is very narrow, within 20 nm range, as compared to Examples 8-11 and FIG. 1, where the size varies from 20 to 100 nm. Again, this attests to the importance of intensive mixing during the particle assembly process. Similar results were obtained with equipment purchased from Bee International, Inc. (South Easton, Mass.). Since the technology is easily scaleable, in terms of pressure and size of equipment, a microfluidics is a convenient technology with which to scale-up the process of nanoparticle production.

### EXAMPLE 13

### Nanoparticle Process and Recovery

Nanoparticles were prepared as in Example 10. After production, the product immediately was filtered via the tangential or cross flow filter (Minimate™ tangential flow filtration capsule, Pall Sciences, Ann Arbor, Mich.). Minimate™ was pretreated with 1% F-68 solution for about 30 minutes prior to product filtration. High recovery (95%), purity via diafiltration and small ion and oligomer removal to near zero and concentration (5-10 time) was achieved. The product is suitable for lyophilization or direct use.

The following references were cited herein:

1. Deplanque et al., Euro. Jour of Cancer 36:1713-1724 (2000).
2. Hagedorn & Bikfaldi, Critical Reviews in Oncology/Hematology 34:89-110 (2000).
3. Jones et al., PPO Update on Principles and Practice of Oncology 14:1-7 (2000).
4. O'Reilly et al., Cell 79:315-328 (1994).
5. O'Reilly et al., Nature Medicine 2:689-692 (1996).
6. O'Reilly et al., Cell 88:277-285 (1997).
7. Streit et al., PNAS USA 96:14888-93 (1999).
8. Steiner, in Angiogenesis: Key Principles-Science, Technology and Medicine, Steiner et al. Eds., Birkhauser, Basel, Switzerland, pp. 449-454 (1992).
9. Pasqualini et al., Cancer Research 60:722-727 (2000).
10. White et al., Hypertension 37 (Pt 2):449-455 (2001).
11. Houston et al., FEBS Letters 492:73-77 (2001).
12. Asai et al., FEBS Letters 510:206-210 (2002).
13. Prokop et al., Advances in Polymer Science 136:52-73 (1998).
14. Bogdanov et al., Neoplasia 1:438-445 (1999).
15. Buckley et al., PNAS USA 82:7340-7344 (1985).
16. Browning et al., J. Exp. Med. 180:1949-1954 (1994).
17. Pasqualini et al., J Cell Biology 130:5 1189-96 (1995).
18. Sephel et al., Matrix Biology 15:263-279 (1996).
19. Sewell et al., Surgery in Gynecology and Obstetrics 100:483 (1955).
20. Spector and Lalor, Biomaterials Science, An Introduction to Materials in Medicine, Ratner et al. eds., Academic Press, pp. 220-228 (1996).
21. Pham et al., Cancer Invest 16:225-230 (1998).
22. Bachhawat-Sikder et al., FEBS Letters 500:75-79 (2001).
23. Plzak et al., Internl. Journal of Oncology 19:59-64 (2001).
24. Liu et al., Jour. of Microelectromechal Systems 9: 190-197 (2000).
25. Nishimura et al., Chemical Engineering Science 48: 1793-1800 (1993).
26. Johnson et al., Amer Inst of Chemical Engineers Journal 49: 2264-2282 (2003).

Any patents or publications mentioned in this specification are indicative of the levels of those skilled in the art to which the invention pertains. Further, these patents and publications are incorporated by reference herein to the same extent as if each individual publication was individually incorporated by reference. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

1. A nanoparticle comprising:
a water-based core comprising:
at least one polyanionic polymer;
a drug or therapeutic peptide; and
a polymer cross-linked to or conjugated to said drug or therapeutic peptide; and
a water-based corona surrounding said core, comprising:
at least one polycationic polymer;
a targeting ligand specific to a cell or tissue of interest; and
a polymer cross-linked to or conjugated to said targeting moiety ligand:
or
a pharmaceutical composition thereof.

2. The nanoparticle of claim 1, further comprising:
a cation in said polycationic corona.

3. The nanoparticle of claim 2, wherein said cation is calcium chloride.

4. The nanoparticle of claim 1, further comprising:
a monovalent or divalent inorganic salt in said polyanionic core.

5. The nanoparticle of claim 4, wherein said salt is sodium chloride or calcium chloride.

6. The nanoparticle of claim 1, further comprising:
a bioluminescent agent or a contrast agent in said polyanionic core.

7. The nanoparticle of claim 6, wherein said bioluminescent agent is luciferase.

8. The nanoparticle of claim 6, wherein said contrast agent is a macromolecular contrast agent or a dynamic contrast enhancing agent.

9. The nanoparticle of claim 1, wherein said polyanionic polymer is high viscosity sodium alginate (SA-HV), low molecular weight sodium alginate (LMW-SA), heparin sulfate, kappa carrageenan, low-esterified pectin (polygalacturonic acid), polyglutamic acid, carboxymethylcellulose, chondroitin sulfate-6, chondroitin sulfate-4, or collagen.

10. The nanoparticle of claim 1, wherein said polycationic polymer is polyvinylamine, spermine hydrochloride, poly(methylene-co-guanidine) hydrochloride, protamine sulfate, polyethyleneimine, polyethyleneimine-ethoxylated, epichlorhydrin modified polyethyleneimine, quartenized polyamide, polydiallyldimethyl ammonium chloride-co-acrylamide, F-68-Pluronic copolymer, or chitosan.

11. The nanoparticle of claim 1, wherein said polyanionic polymers are high viscosity sodium alginate, cellulose sulfate, said nanoparticle further comprising sodium chloride in the core; and said polycationic polymers are spermine hydrochloride, poly(methylene-co-guanidine) hydrochloride and F-68 Pluronic copolymer, said nanoparticle further comprising calcium chloride in the corona.

12. The nanoparticle of claim 1, wherein said polyanionic polymers are high viscosity sodium alginate and cellulose sulfate, said nanoparticle further comprising heparin and calcium chloride in the core and said polycationic polymers are spermine hydrochloride, poly(methylene-co-guanidine) hydrochloride and F-68 Pluronic copolymer, said nanoparticle further comprising calcium chloride in the corona.

13. The nanoparticle of claim 1, wherein said polyanionic polymers and said polycationic polymers have a low molecular weight.

14. The nanoparticle of claim 13, wherein said low molecular weight polyanionic polymers are LMW sodium alginate, LMW sodium hyaluronate, pentasodium tripolyphosphate, heparin sulfate or chondroitin sulfate.

15. The nanoparticle of claim 13, wherein said low molecular weight polycationic polymers are LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, LMW chitosan, or pluronic F-68.

16. The nanoparticle of claim 13, wherein said LMW polyanionic polymers are chondroitin-6-sulfate and heparin sulfate and said polycationic polymers are spermine hydrochloride, poly(methylene-co-guanidine) hydrochloride and F-68 Pluronic copolymer.

17. The nanoparticle of claim 16, wherein said LMW polycationic polymers are spermine hydrochloride and F-68 Pluronic copolymer.

18. The nanoparticle of claim 13, wherein said polyanionic polymers are LMW sodium alginate and heparin sulfate and said polycationic polymers are spermine hydrochloride, poly(methylene-co-guanidine) hydrochloride and F-68, said nanoparticle further comprising calcium chloride in the corona.

19. The nanoparticle of claim 18, wherein said polyanionic polymer is LMW sodium alginate.

20. The nanoparticle of claim 13, wherein said LMW polyanionic polymers are LMW sodium alginate and heparin sulfate and said polycationic polymers are spermine hydrochloride, chitosan and F-68.

21. The nanoparticle of claim 1, wherein said cross-linking or conjugating core polymer is dextran polyaldehyde, LMW sodium alginate or heparin sulfate.

22. The nanoparticle of claim 1, wherein said drug or therapeutic peptide is a growth factor, a gene, angiostatin, endostatin, thrombospondin 1 or a peptide fragment thereof, or thrombospondin 2 or a peptide fragment thereof or a combination thereof.

23. The nanoparticle of claim 1, wherein said cross-linking or conjugating corona polymer is dextran polyaldehyde or activated polyethylene glycol.

24. The nanoparticle of claim 1, wherein said targeting ligand is TSP517, TSP521, apoE, a polysaccharide targeted to lectin or lectin targeted to a glycan.

25. A method of delivering a drug or therapeutic peptide to a cell or tissue of interest in an individual, comprising:
administering nanoparticles of claim 1 comprising the drug or therapeutic peptide to said individual; and
targeting said nanoparticles to the cell or tissue via the targeting ligand comprising said nanoparticles, thereby delivering said drug or therapeutic protein to the cell or tissue in the individual.

26. The method of claim 25, further comprising:
imaging said cell or tissue, wherein said nanoparticles comprise a bioluminescent agent or contrast agent in said polyanionic core.

27. The method of claim 26, wherein said bioluminescent agent is luciferase.

28. The method of claim 26, wherein said contrast agent is a macromolecular contrast agent or a dynamic contrast enhancing agent.

29. The method of claim 25, wherein said cell or tissue of interest comprises tumor vasculature.

30. A method of imaging a cell or tissue of interest in an individual during delivery of a drug or therapeutic peptide thereto, comprising:
administering nanoparticles of claim 6 comprising the drug or therapeutic peptide to said individual;
targeting said nanoparticles to the cell or tissue via the targeting ligand comprising said nanoparticles; and
simultaneously imaging said cell or tissue via the bioluminescent agent or contrast agent comprising the core of said nanoparticles as said drug or therapeutic peptide is delivered, thereby imaging said cell or tissue of interest in the individual during delivery thereof.

31. The method of claim 30, wherein said tissue is tumor vasculature.

32. A method of producing a nanoparticle suitable for delivery of a drug or therapeutic protein to a cell or tissue of interest in an individual, comprising:
mixing at least one stream of a solution comprising components of the polyanionic core of the nanoparticle of claim 1 with at least one stream of a solution comprising the components of the polycationic corona of the nanoparticle of claim 1; and
forming nanoparticles having a complex multipolymeric structure to crosslink or conjugate the drug or therapeutic protein comprising said core therewithin and to crosslink or conjugate the targeting ligand comprising said corona thereto; wherein the complex structure of said nanoparticle is suitable to deliver the drug or therapeutic peptide to the cell or tissue of interest.

33. The method of claim 32, further comprising:
adding a cation to said corona solution.

34. The method of claim 33, wherein said cation is present in said corona solution at a concentration of about 0.1 wt-% to about 1 wt-%.

35. The method of claim 33, wherein said cation is calcium chloride.

36. The method of claim 32, further comprising:
adding a monovalent or divalent inorganic salt to said core solution.

37. The method of claim 36, wherein said salt is present in said core solution at a concentration of about 0.5 wt-% to about 2 wt-%.

38. The method of claim 36, wherein said salt is sodium chloride or calcium chloride.

39. The method of claim 32, further comprising:
adding a bioluminescent agent or contrast agent to said core solution.

40. The method of claim 39, wherein said bioluminescent agent is luciferase.

41. The method of claim 39, wherein said contrast agent is a macromolecular contrast agent or a dynamic contrast enhancing agent.

42. The method of claim 32, said mixing step comprising:
simple flowing of one stream of said core solution and one stream of said corona solution together in a batch mode; and
stirring the mixed solutions.

43. The method of claim 32, said mixing step comprising:
laminar flowing of one or more streams each of said core solution and of said corona solution together in a continuous mode.

44. The method of claim 43, wherein the laminar flow of at least one of said streams is oscillated.

45. The method of claim 44, wherein said stream(s) is oscillated at a frequency of about 5 Hz and 200 Hz.

46. The method of claim 43, wherein laminar flow of said streams is pressurized.

47. The method of claim 46, wherein said streams are pressurized independently up to about 200,000 psi.

48. The method of claim 32, further comprising:
independent feedback monitoring in real time of a characteristic of said nanoparticle or of said process or a combination thereof, said characteristic comprising nanoparticle size, nanoparticle charge density, flow rates of streams, flow ratios, pH, salt content, or ethanol content; and
optimizing said characteristic in real time.

49. The method of claim 32, wherein said solutions are mixed at a flow ratio of about 1:1 to about 1:12 polyanion:polycation polymers.

50. The method of claim 32, further comprising:
washing said nanoparticles.

51. The method of claim 50, further comprising:
cryoprotecting said nanoparticles in a cryopreservation solution; and
lyophilizing said cryoprotected nanoparticles.

52. The method of claim 32, wherein said core polymers individually are present in a concentration of about 0.01 wt-% to about 0.5 wt-%.

53. The method of claim 32, wherein said corona polymers individually are present in a concentration of about 0.01 wt-% to about 5.0 wt-%.

54. The method of claim 32, wherein said drug is present in a concentration of about 0.03 wt-% to about 0.4 wt-%.

55. The method of claim 32, wherein said targeting ligand is present in a concentration about 0.01 wt-% to about 5.0 wt-%.

56. A nanoparticle comprising:
a water-based core comprising:
HV sodium alginate and cellulose sulfate; and
a drug or therapeutic peptide crosslinked with dextran polyaldehyde, said core further comprising calcium chloride; or
a drug or therapeutic peptide conjugated to heparin sulfate, said core further comprising sodium chloride; and
a water-based corona surrounding said core, comprising:
spermine hydrochloride, poly(methylene-co-guanidine) hydrochloride and pluronic F-68;
calcium chloride; and
a targeting ligand conjugated to an activated polyethylene glycol or crosslinked to dextran polyaldehyde;
or a pharmaceutical composition thereof.

57. The nanoparticle of claim 56, further comprising:
a bioluminescent agent or contrast agent in said polyanionic core.

58. The nanoparticle of claim 57, wherein said bioluminescent agent is luciferase.

59. The nanoparticle of claim 57, wherein said contrast agent is a macromolecular contrast agent or a dynamic contrast enhancing agent.

60. The nanoparticle of claim 56, wherein said drug or therapeutic peptide is a growth factor, a gene, angiostatin, endostatin, thrombospondin 1 or a peptide fragment thereof, or thrombospondin 2 or a peptide fragment thereof or a combination thereof.

61. The nanoparticle of claim 56, wherein said targeting ligand is TSP517, TSP521, apoE, a polysaccharide targeted to lectin or lectin targeted to a glycan.

62. A nanoparticle comprising:
a water-based core comprising:
at least one LMW polyanionic polymer; and
a drug or therapeutic peptide crosslinked with dextran polyaldehyde; or
a drug or therapeutic peptide conjugated to heparin sulfate or LMW sodium alginate; and
a water-based corona surrounding said core, comprising:
at least one LMW polycationic polymer; and
a targeting ligand conjugated to an activated polyethylene glycol or crosslinked to dextran polyaldehyde;
or a pharmaceutical composition thereof.

63. The nanoparticle of claim 62, further comprising:
a monovalent or divalent inorganic salt in said core.

64. The nanoparticle of claim 63, wherein said inorganic salt is sodium chloride or calcium chloride.

65. The nanoparticle of claim 62, further comprising:
a cation in said corona.

66. The nanoparticle of claim 65, wherein said cation is calcium chloride.

67. The nanoparticle of claim 62, further comprising:
a bioluminescent agent or contrast agent in said polyanionic core.

68. The nanoparticle of claim 67, wherein said bioluminescent agent is luciferase.

69. The nanoparticle of claim 67, wherein said contrast agent is a macromolecular contrast agent or a dynamic contrast enhancing agent.

70. The nanoparticle of claim 62, wherein said drug or therapeutic peptide is a growth factor, a gene, angiostatin, endostatin, thrombospondin 1 or a peptide fragment thereof, or thrombospondin 2 or a peptide fragment thereof or a combination thereof.

71. The nanoparticle of claim 62, wherein said targeting ligand is TSP517, TSP521, apoE, a polysaccharide targeted to lectin or lectin targeted to a glycan.

72. The nanoparticle of claim 62, wherein said LMW polyanionic polymers are chondroitin-6-sulfate and heparin sulfate and said polycationic polymers are spermine hydrochloride, poly(methylene-co-guanidine) hydrochloride and F-68 Pluronic copolymer.

73. The nanoparticle of claim 72, wherein said LMW polycationic polymers are spermine hydrochloride and F-68 Pluronic copolymer.

74. The nanoparticle of claim 62, wherein said LMW polyanionic polymers are LMW sodium alginate and heparin sulfate and said polycationic polymers are spermine hydrochloride, poly(methylene-co-guanidine) hydrochloride and F-68, said nanoparticle further comprising calcium chloride in the corona.

75. The nanoparticle of claim 74, wherein said LMW polyanionic polymer is LMW sodium alginate.

76. The nanoparticle of claim 62, wherein said LMW polyanionic polymers are LMW sodium alginate and heparin sulfate and said polycationic polymers are spermine hydrochloride, chitosan and F-68.

77. A nanoparticle comprising:
a water-based core comprising:
at least one polymer having a low molecular weight;
a drug or therapeutic peptide; and
a polymer cross-linked to or conjugated to said drug or therapeutic peptide; and
a water-based corona surrounding said core, comprising:
at least one polymer having a low molecular weight of opposite charge to said low molecular weight core polymer(s);
a targeting ligand specific to a cell or tissue of interest; and
a polymer cross-linked to or conjugated to said targeting ligand; or
a pharmaceutical composition thereof.

78. The nanoparticle of claim 77, further comprising:
a cation in said polycationic corona.

79. The nanoparticle of claim 78, wherein said cation is calcium chloride.

80. The nanoparticle of claim 77, further comprising:
a monovalent or divalent inorganic salt in said polyanionic core.

81. The nanoparticle of claim 80, wherein said salt is sodium chloride or calcium chloride.

82. The nanoparticle of claim 77, further comprising:
a bioluminescent agent or a contrast agent in said polyanionic core.

83. The nanoparticle of claim 82, wherein said bioluminescent agent is luciferase.

84. The nanoparticle of claim 82, wherein said contrast agent is a macromolecular contrast agent or a dynamic contrast enhancing agent.

85. The nanoparticle of claim 77, wherein said core polymers or said corona polymers are LMW sodium alginate, LMW sodium hyaluronate, pentasodium tripolyphosphate, heparin sulfate or chondroitin sulfate.

86. The nanoparticle of claim 77, wherein said core polymers or said corona polymers are LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, LMW chitosan, or pluronic F-68.

87. The nanoparticle of claim 77, wherein said core polymers are chondroitin-6-sulfate and heparin sulfate and said corona polymers are spermine hydrochloride, poly(methylene-co-guanidine) hydrochloride and F-68 Pluronic copolymer.

88. The nanoparticle of claim 87, Wherein said corona polymers are spermine hydrochloride and F-68 Pluronic copolymer.

89. The nanoparticle of claim 77, wherein said core polymers are LMW sodium alginate and heparin sulfate and said corona polymers are spermine hydrochloride, poly(methylene-co-guanidine) hydrochloride and F-68, said nanoparticle further comprising calcium chloride in the corona.

90. The nanoparticle of claim 89, wherein said core polymer is LMW sodium alginate.

91. The nanoparticle of claim 77, wherein said core polymers are LMW sodium alginate and heparin sulfate and said corona polymers are spermine hydrochloride, chitosan and F-68.

92. The nanoparticle of claim 77, wherein said cross-linking or conjugating core polymer is dextran polyaldehyde, LMW sodium alginate or heparin sulfate.

93. The nanoparticle of claim 77, wherein said drug or therapeutic peptide is a growth factor, a gene, angiostatin, endostatin, thrombospondin 1 or a peptide fragment thereof, or thrombospondin 2 or a peptide fragment thereof or a combination thereof.

94. The nanoparticle of claim 77, wherein said cross-linking or conjugating corona polymer is dextran polyaldehyde or activated polyethylene glycol.

95. The nanoparticle of claim 77, wherein said targeting ligand is TSP517, TSP521, apoE, a polysaccharide targeted to lectin or lectin targeted to a glycan.

96. A method of producing low molecular weight nanoparticles suitable for delivery of a drug or therapeutic protein to a cell or tissue of interest in an individual, comprising:
laminar flowing of one or more streams of a solution comprising the components of the nanoparticle core of claim 77 with one or more streams of a solution comprising the components of the nanoparticle corona of claim 81 together in a continuous mode; and
forming nanoparticles having a complex multipolymeric structure to crosslink or conjugate the drug or therapeutic protein comprising said core therewithin and to crosslink or conjugate the targeting ligand comprising said corona thereto; wherein the complex structure of said nanoparticle is suitable to deliver the drug or therapeutic peptide to the cell or tissue of interest.

97. The method of claim 96, wherein the laminar flow of at least one of said streams is oscillated.

98. The method of claim 97, wherein said stream(s) is oscillated at a frequency of about 5 Hz and 200 Hz.

99. The method of claim 96, wherein laminar flow of said streams is pressurized.

100. The method of claim 99, wherein said streams are pressurized independently up to about 200,000 psi.

101. The method of claim 96, further comprising:
independent feedback monitoring in real time of a characteristic of said nanoparticle or of said process or a combination thereof, said characteristic comprising nanoparticle size, nanoparticle charge density, flow rates of streams, flow ratios, pH, salt content, or ethanol content; and
optimizing said characteristic(s) in real time.

102. The method of claim 96, further comprising:
washing said nanoparticles.

103. The method of claim 102, further comprising:
cryoprotecting said nanoparticles in a cryopreservation solution; and
lyophilizing said cryoprotected nanoparticles.

104. The method of claim 96, further comprising:
adding a cation to said corona solution.

105. The method of claim 104, wherein said cation is present in said corona solution at a concentration of about 0.1 wt-% to about 1 wt-%.

106. The method of claim 104, wherein said cation is calcium chloride.

107. The method of claim 96, further comprising:
adding a monovalent or divalent inorganic salt to said core solution.

108. The method of claim 107, wherein said salt is present in said core solution at a concentration of about 0.5 wt-% to about 2 wt-%.

109. The method of claim 107, wherein said salt is sodium chloride or calcium chloride.

110. The method of claim 96, further comprising:
adding a bioluminescent agent or contrast agent to said core solution.

111. The method of claim 110, wherein said bioluminescent agent is luciferase.

112. The method of claim 110, wherein said contrast agent is a macromolecular contrast agent or a dynamic contrast enhancing agent.
